(19) United States
(12) Patent Application Publication
(10) Pub. No.: US 2012/0172716 A1
(43) Pub. Date: Jul. 5, 2012

(54) ELECTROMAGNETIC COIL SENSOR FOR A MEDICAL DEVICE

(76) Inventors: Ran Sela, Tel Aviv-Yafo (IL); Lior Sobe, Kadima (IL); Dan Seter, Haifa (IL)

(21) Appl. No.: 12/982,120

(22) Filed: Dec. 30, 2010

(51) Int. Cl. A61B 5/05; H01F 7/127

(52) U.S. Cl. 600/424; 29/605

(57) ABSTRACT

A positioning sensor for use in a medical device wherein the device has a heat-fused layer comprising a first material having a first melting temperature. The sensor has a tubular core comprising a second material having a second melting temperature that is higher than the first temperature. The core has a central through-bore extending along an axis between opposing axial ends of the core, and where the core further has a radially-outermost outermost winding surface. The sensor includes an electrically conductive coil wound on the winding surface.

### BACKGROUND OF THE INVENTION

a. Field of the Invention

The present disclosure relates to a electromagnetic positioning sensor for a medical device and a method for mounting an electromagnetic positioning sensor on a medical device.

b. Background Art

Medical devices such as guidewires, catheters, introducers and the like with electromagnetic coil position sensors for device navigation are used in various medical procedures in the body. Assembling electromagnetic coil sensors within the space constraints of a medical device without sacrificing sensitivity presents various design and manufacturing challenges. One such challenge is including a sensor in a device manufacturing process that can survive a high temperature fusion step, for example as is conventional for reflow of an outer polymer (e.g., PEBAX) jacket. It is thus essential that the design of the sensor account for the mechanical forces, material state transitions, and dimensional changes that may occur during high-temperature fusion.

There are additional design considerations, beyond manufacturability, when incorporating position sensors into medical device designs, for example, dimensional considerations. For example, it is known to provide a so-called “over-the-wire” type of medical catheter where the catheter includes an inner, longitudinally-extending central lumen for a guidewire, as seen by reference to U.S. Patent Publication 2004/0097804 to Sobe entitled “METHOD AND SYSTEM FOR MOUNTING AN MPS SENSOR ON A CATHETER,” owned by the common assignee of the present invention and hereby incorporated by reference in its entirety. Such a medical catheter may be further configured to be contained within a sheath or introducer. The resulting configuration presents further challenges when incorporating one or more coil position sensors into its design. First, incorporation of one or more position sensors must not enlarge the outside diameter (O.D.) of the device (i.e., its radial size) since the device must still be able to fit within the large central lumen of the above-mentioned outer sheath or introducer. Second, incorporation of one or more position sensors must not block or otherwise impair the central lumen, which must be keep open and otherwise unrestricted for the guidewire.

There is therefore a need for a medical device that incorporates coil position sensors that minimizes or eliminates one or more of the problems set forth above.

### BRIEF SUMMARY OF THE INVENTION

A positioning sensor for use in a medical device wherein the device has a heat-fused layer comprising a first material having a first melting temperature associated therewith, comprises: a tubular core comprising a second material having a second melting temperature associated therewith that is higher than said first temperature; said core having a central through-bore extending along an axis between opposing axial ends of said core, said core further having a radially-outermost winding surface; and an electrically conductive coil wound on said winding surface.

A medical device configured for use with a medical positioning system (MPS) comprises: an elongate body having an axis and an outer surface, said outer surface including a plurality of axially spaced circumferentially-extending grooves, each of the grooves configured to receive a respective positioning sensor, the body having an outside diameter taken with respect to the outer surface; and a plurality of sensors respectively disposed in a corresponding one of the grooves, the sensors each comprising a respective coil, each one of the sensors being configured relative to a corresponding one of the grooves so as to remain within an envelope defined by the outside diameter.

### BRIEF DESCRIPTION OF THE DRAWINGS

FIG. 1 is a schematic and block diagram view of a system incorporating an embodiment of a position-sensing medical device.

FIG. 2 is a diagrammatic view of the system of FIG. 1 in a catheter-lab environment.

FIG. 3 is an isometric view of a first embodiment of the present invention.

FIG. 4 is a cross-sectional view of the first embodiment taken substantially along line 4-4 in FIG. 3.

FIGS. 5-8 are cross-sectional side views of a reflow mandrel assembly in various stages of build-up in a method of manufacture of a second embodiment of the present invention.

FIG. 9 is a cross-sectional end view of the second embodiment, taken substantially along line 9-9 in FIG. 8 and showing the full circumference of the second embodiment.

FIG. 10 is an isometric view of a third embodiment of the present invention.

FIG. 11 is a cross-sectional side view of the third embodiment, taken substantially along line 11-11 in FIG. 10.

FIG. 12 is a cross-sectional end view of the third embodiment, taken substantially along line 12-12 in FIG. 10.

FIG. 13 is a schematic and block diagram view of one exemplary embodiment of a medical positioning system (MPS) as shown in block form in FIG. 1.

### DETAILED DESCRIPTION OF THE INVENTION

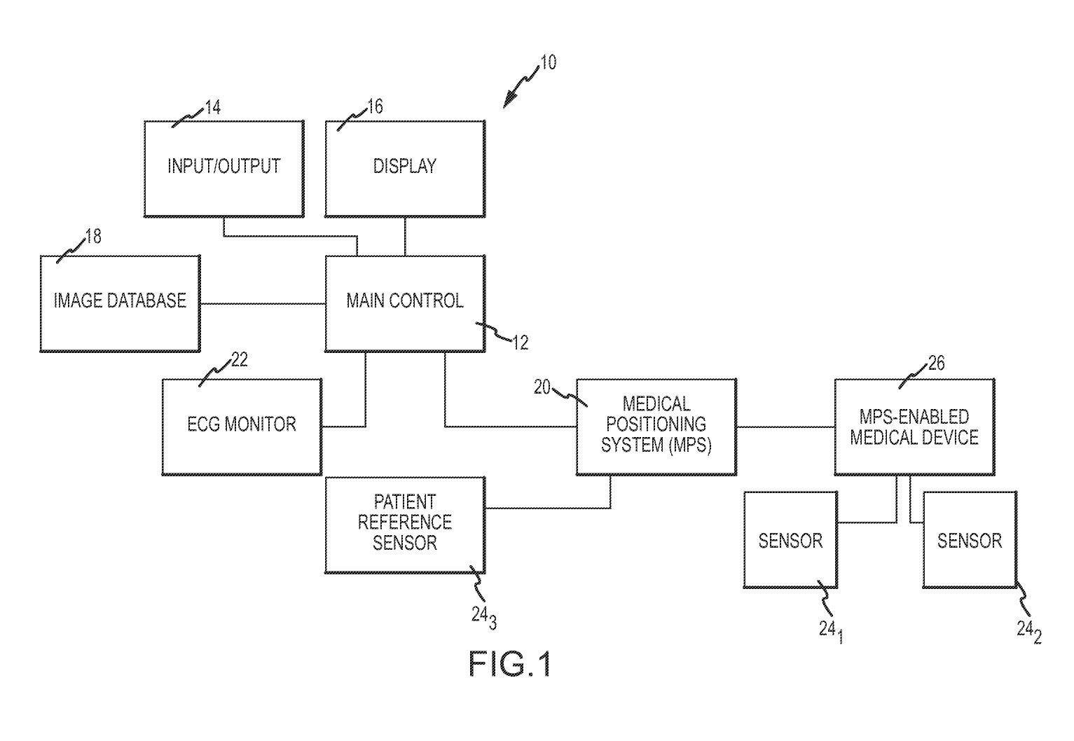

Referring now to the drawings wherein like reference numerals are used to identify identical components in the various views, FIG. 1 is a diagrammatic view of a system 10 in which a position sensing medical device such as a guidewire or catheter may be used. It should be understood that while embodiments will be described in connection with a catheter-lab environment, this is exemplary only and not limiting in nature.

Before proceeding to a detailed description of the several medical device embodiments of the present invention, a description of an exemplary environment in which such medical device embodiments may be used will be first set forth. With continued reference to FIG. 1, the system 10 as depicted includes a main electronic control unit 12 (e.g., a processor) having various input/output mechanisms 14, a display 16, an optional image database 18, a localization system such as a medical positioning system (MPS) 20, an electrocardiogram (ECG) monitor 22, one or more MPS location sensors respectively designated 24₁, 24₂, and 24₃ (i.e., shown as a patient reference sensor), and an MPS-enabled elongate medical device 26 which itself includes one or more of the above-described MPS location sensors, shown in exemplary fashion as having two such sensors 24₁ and 24₂.

Input/output mechanisms 14 may comprise conventional apparatus for interfacing with a computer-based control unit, for example, a keyboard, a mouse, a tablet, a foot pedal, a switch or the like. Display 16 may also comprise conventional apparatus.

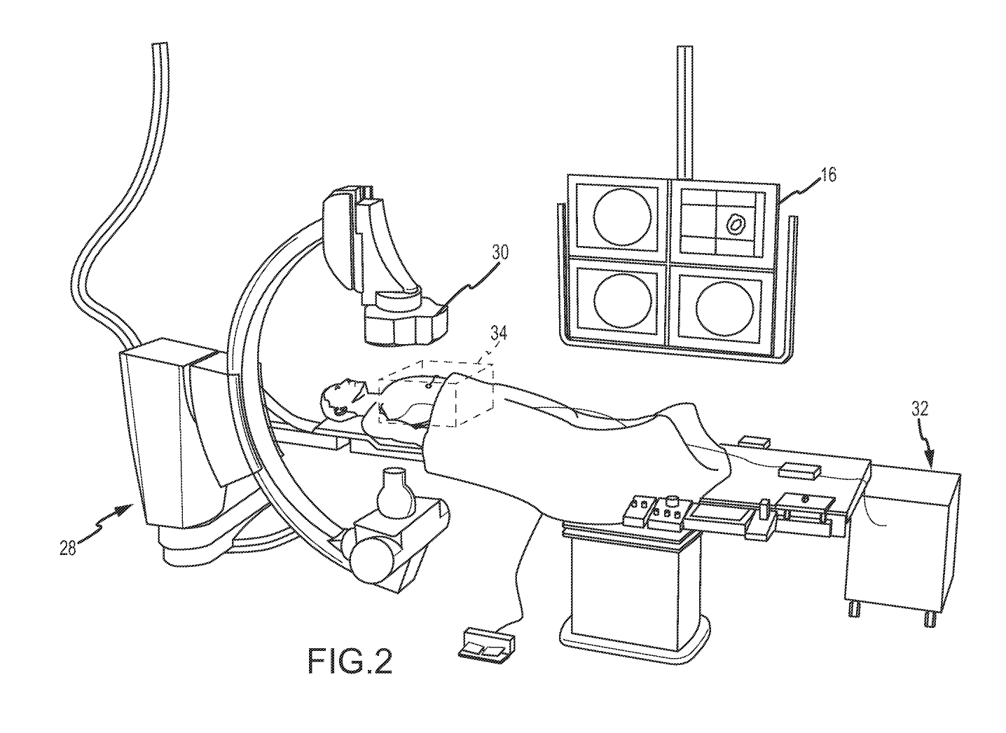

Embodiments consistent with the invention may find use in navigation applications that use imaging of a region of interest. Therefore system 10 may optionally include image database 18. Image database 18 may be configured to store image information relating to the patient's body, for example a region of interest surrounding a destination site for medical device 26 and/or multiple regions of interest along a navigation path contemplated to be traversed by device 26 to reach the destination site. The image data in database 18 may comprise known image types including (1) one or more two-dimensional still images acquired at respective, individual times in the past; (2) a plurality of related two-dimensional images obtained in real-time from an image acquisition device (e.g., fluoroscopic images from an x-ray imaging apparatus, such as that shown in exemplary fashion in FIG. 2) wherein the image database acts as a buffer (live fluoroscopy); and/or (3) a sequence of related two-dimensional images defining a cine-loop (CL) wherein each image in the sequence has at least an ECG timing parameter associated therewith adequate to allow playback of the sequence in accordance with acquired real-time ECG signals obtained from ECG monitor 22. It should be understood that the foregoing are examples only and not limiting in nature. For example, the image database may also include three-dimensional image data as well. It should be further understood that the images may be acquired through any imaging modality, now known or hereafter developed, for example X-ray, ultra-sound, computerized tomography, nuclear magnetic resonance or the like.

MPS 20 is configured to serve as the localization system and therefore to determine positioning (localization) data with respect to one or more of MPS location sensors 24ᵢ (where i=1 to n) and output a respective location reading. The location readings may each include at least one or both of a position and an orientation (P&O) relative to a reference coordinate system, which may be the coordinate system of MPS 20. For example, the P&O may be expressed as a position (i.e., a coordinate in three axes X, Y and Z) and orientation (i.e., an azimuth and elevation) of a magnetic field sensor in a magnetic field relative to a magnetic field generator(s) or transmitter(s).

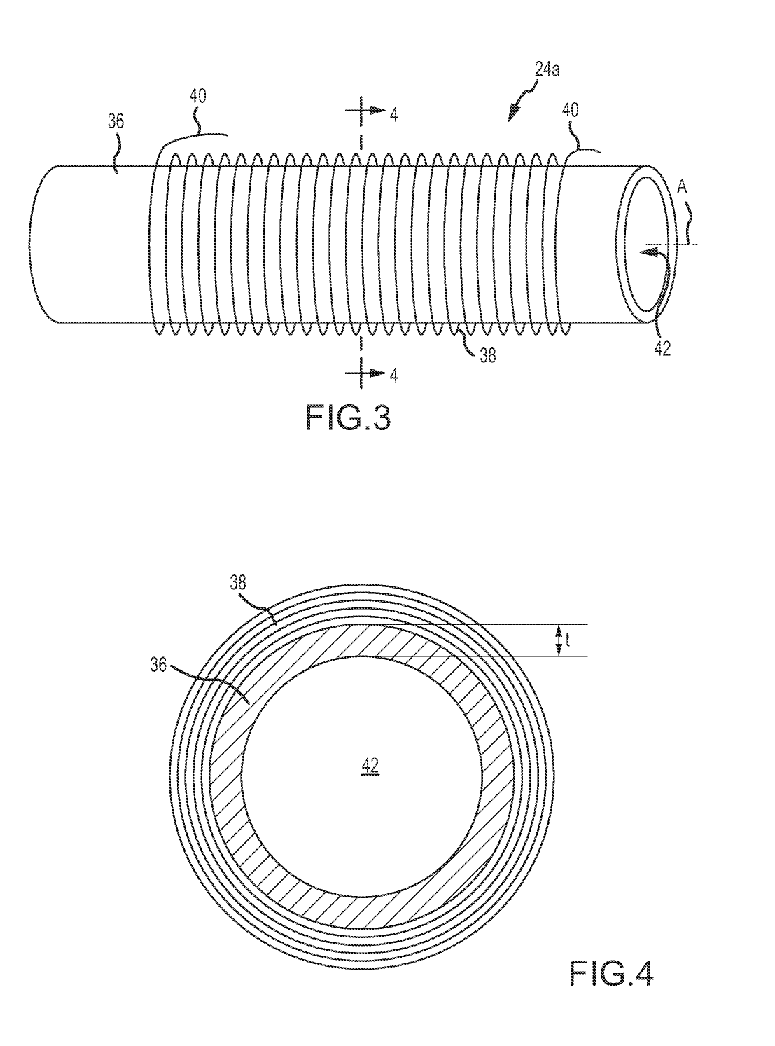

MPS 20 determines respective locations (i.e., P&O) in the reference coordinate system based on capturing and processing signals received from the magnetic field sensors 24ᵢ while such sensors are disposed in a controlled low-strength AC magnetic field (see FIG. 2). From an electromagnetic perspective, these sensors develop a voltage that is induced on the coil residing in a changing magnetic field, as contemplated here. Sensors 24ᵢ are thus configured to detect one or more characteristics of the magnetic field(s) in which they are disposed and generate an indicative signal, which is further processed by MPS 20 to obtain a respective P&O thereof. Exemplary design features and manufacturing processes and methods for sensors 24ᵢ and medical devices incorporating such sensors will be described in greater detail below in conjunction with FIGS. 3-12.

At least one of MPS sensors 24₁ and 24₂, both in one embodiment, and optionally additional MPS sensors in further embodiments, may be associated with MPS-enabled medical device 26. Another MPS sensor, namely, patient reference sensor (PRS) 24₃ (if provided in system 10) is configured to provide a positional reference of the patient's body so as to allow motion compensation for gross patient body movements and/or respiration-induced movements. PRS 24₃ may be attached to the patient's manubrium sternum, a stable place on the chest, or other location that is relatively positionally stable. Like MPS location sensors 24₁ and 24₂, PRS 24₃ is configured to detect one or more characteristics of the magnetic field in which it is disposed wherein MPS 20 provides a location reading (e.g., a P&O reading) indicative of the PRS's position and orientation in the reference coordinate system.

The electro-cardiogram (ECG) monitor 22 is configured to continuously detect an electrical timing signal of the heart organ through the use of a plurality of ECG electrodes (not shown), which may be externally-affixed to the outside of a patient's body. The timing signal generally corresponds to the particular phase of the cardiac cycle, among other things. Generally, the ECG signal(s) may be used by the control unit 12 for ECG synchronized play-back of a previously captured sequence of images (cine loop) stored in database 18. ECG monitor 22 and ECG-electrodes may both comprise conventional components.

FIG. 2 is a diagrammatic view of system 10 as incorporated into an exemplary catheter laboratory. System 10 is shown as being incorporated into a fluoroscopic imaging system 28, which may include commercially available fluoroscopic imaging components (i.e., “Catheter Lab”). MPS 20 includes a magnetic transmitter assembly (MTA) 30 and a magnetic processing core 32 for determining location (P&O) readings. MTA 30 is configured to generate the magnetic field(s) in and around the patient's chest cavity, in a predefined three-dimensional space identified as a motion box 34. MPS sensors 24ᵢ as described above are configured to sense one or more characteristics of the magnetic field(s) and when the sensors are in motion box 34, each generate a respective signal that is provided to magnetic processing core 32. Processing core 32 is responsive to these detected signals and is configured to calculate respective P&O readings for each MPS sensor 24ᵢ in motion box 34. Thus, MPS 20 enables real-time tracking of each sensor 24ᵢ in three-dimensional space.

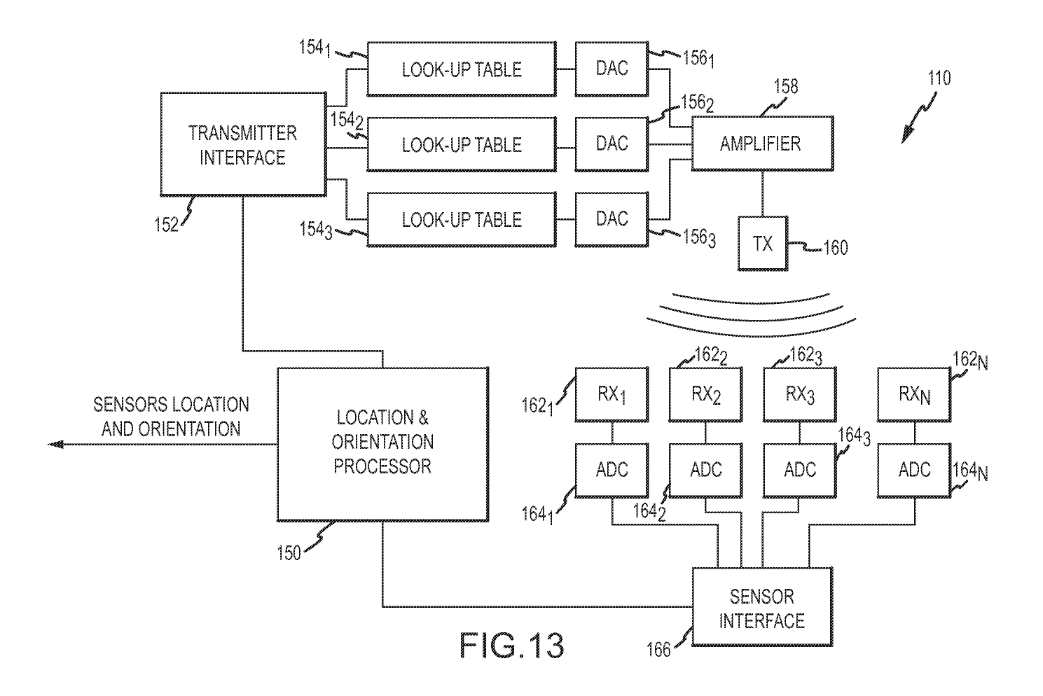

The positional relationship between the image coordinate system and the MPS reference coordinate system may be calculated based on a known optical-magnetic calibration of the system (e.g., established during setup), since the positioning system and imaging system may be considered fixed relative to each other in such an embodiment. However, for other embodiments using other imaging modalities, including embodiments where the image data is acquired at an earlier time and then imported from an external source (e.g., imaging data stored in database 18), a registration step registering the MPS coordinate system and the image coordinate system may need to be performed so that MPS location readings can be properly coordinated with any particular image being used. One exemplary embodiment of an MPS 20 will be described in greater detail below in connection with FIG. 13.

In sum, medical devices incorporating electromagnetic field coil position sensors provide enhanced capabilities when used in connection with a compatible localization system, such as MPS 20. For example, the P&O information from MPS 20 allows a representation of the MPS-equipped medical device (e.g., at least the distal tip portion) to be superimposed on images of the region of interest, thereby reducing the use of fluoroscopy (including patient exposure to X-rays) as well as reducing or eliminating the use of dyes to enhance visibility in fluoroscopic images.

FIG. 3 is an isometric view of a first embodiment of a positioning sensor configured for use in medical device such as device 26, herein designated positioning sensor 24a. Sensor 24a includes a sensor core 36 and a sensor coil 38. Sensor core 36 may be an elongated hollow tube with a central axis (shown as “A”) and a central through-bore 42 extending between opposing axial ends. Bore 42 is configured to allow sensor 24a to be threaded on or applied to medical devices. Coil 38 may be wound on the radially outermost surface—the winding surface—of core 36 with the free coil ends 40 being left exposed for use as leads in connecting coil 38 to MPS 20. Coil 38 comprises electrically conductive material and may comprise conventional wire having suitable characteristics, such as material or alloy type, thickness (wire gauge—AWG), insulative coating type and thickness, and the like, all as known in the art. In addition, coil 38 may include a predetermined number of turns wound in a winding pattern suitable for detecting characteristics of the electromagnetic field(s) in which the medical device carrying such a sensor is expected to be used.

The sensor 24a may be incorporated in an assembly for making a medical device in an intermediate stage of manufacture, which assembly may thereafter be finished with an outer layer (i.e., radially-outwardly from core 36 and coil 38) that may be heat-fused, e.g., in a reflow lamination process. For example, such a heat-fused outer layer may comprise an elastomer commercially available under the trade designation PEBAX® from Arkema, Inc, with a melting temperature of about 130-175° C. The outer layer may be the radially-outermost layer of the finished device, or it may be an intermediate layer, but still radially-outwardly of the sensor 24a. Reflow may be conducted at a temperature of about 450° F. Core 36 may comprise a material such as, for example only, a polymer, such as polyimide, or metal, which material may withstand typical heat-fusing temperatures—i.e., the material may be associated with a high melting point—so that core 36 does not deform or disintegrate during reflow. For example, core 36 may comprise a polymer material, such as polyimide, that maintains its structural integrity for temperatures exceeding 450° F. (i.e., has a melting temperature higher than 450° F.). Materials for core 36 may also be selected for, among other things, their magnetic permeability to enhance the position sensor sensitivity, or for the similarity of their mechanical properties to the desired mechanical properties of the final medical device. For example, a metal core may be more desirable to increase sensitivity in a smaller-diameter sensor (e.g., for use in a guidewire application). Because the respective melting temperatures associated with the one or more materials included in core 36 may be relatively higher as compared to the melting temperature of the above-mentioned device outer layer, core 36 is capable of maintaining structural integrity despite the exposure to heat attendant the reflow lamination process.

FIG. 4 is a cross-sectional view of the sensor 24a taken substantially along line 4-4 in FIG. 3. Core 36 has a wall thickness, t, which may be about 60 micrometers or less for certain materials, such as polyimide, with tight tolerances of up to 20 micrometers, or any dimensions required by design or manufacturing constraints. Central through-bore 42 may be relatively large, enabling sensor 24a to be threaded on a medical device. Thickness t may be increased or decreased as size constraints of the final medical device require.

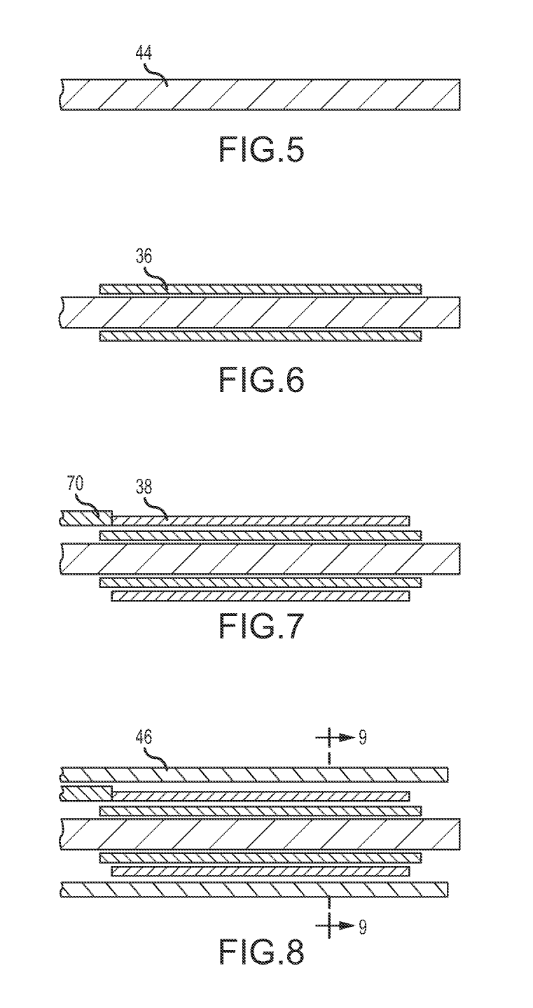

In one embodiment, sensor 24a is manufactured separately and apart from the manufacture of the medical device in which sensor 24a will be ultimately incorporated. In this embodiment, coil 38 may be temporarily (or permanently) fixed both to itself and to core 36, using conventional approaches for example. At least one electrical wire 70 (best shown in FIGS. 7-8) may also be included in the separate assembly of sensor 24a, which wiring 70 may be coupled to coil 38 and also fixed to coil 38 and core 36. In another embodiment, however, as described below in connection with FIGS. 5-8, the sensor 24a is directly included in the manufacture of the medical device.

FIGS. 5-8 are cross-sectional, exaggerated side views of a reflow mandrel assembly in various stages of build-up in a method of manufacture of a medical device that directly incorporates sensor 24a during manufacture (as opposed to separately making the sensor 24a for later incorporation). FIGS. 5-8 show a heat-fused jacket on the sensor itself, in addition to any additional jacket or outer layers that may cover the medical device in which sensor 24a is incorporated. It should be understood that FIGS. 5-8 show the distal end portion of the medical device where a positioning sensor 24a is typically disposed. As used with reference to a medical device, “distal” refers to an end that is advanced to the region of interest within a body while “proximal” refers to the opposite end that is disposed outside of the body and manipulated manually by a clinician or automatically through, for example, robotic controls. It should be further understood that while radial “gaps” or clearances are shown in FIGS. 5-8 between the several layers of materials, this is done for clarity only to distinguish the separate layers.

FIG. 5 shows a mandrel 44 which may be circular in radial cross-section and have a desired length, in view of the elongate medical device to be made.

As shown in FIG. 6, core 36 may be provided over mandrel 44. As described in connection with the first embodiment, core 36 may comprise a material associated with a relatively high melting point, such as, for example only, polyimide or another polymer, so it can withstand a heat fusion process without substantial deformation or disintegration. Core 36 may be tubular in shape, with a central through-bore extending along the core's central axis between opposing axial ends, and have a length, inner diameter, outer diameter, and thickness necessary for a particular application of sensor 24a. The outer diameter of core 36 may be defined by the its outermost surface, which may act as a winding surface.

FIG. 7 shows the assembly after coil 38 has been wound on the outermost surface of core 36. As in the first embodiment, coil 38 is not limited in number, direction, pitch, or angle of windings, or in the dimensions or material of the wire that is wound to form coil 38. Coil 38 may be coupled to at least one axially-proximally-extending electrical wire 70 for connectivity within the medical device (e.g., connection from the distal end where the sensor 24a is located to the device proximal end, where a connector or the like is typically provided—the connector being configured for connection to MPS 20 and potentially other external apparatus, depending on the nature and function of the medical device). Wiring 70 may comprise an unshielded twisted-pair cable or another electrical signal or power cable known in the art.

As shown in FIG. 8, an outer layer 46 is then applied over (i.e., radially-outwardly of) the sub-assembly thus formed and further over portions of the medical device proximal of the sub-assembly (not shown). Outer layer 46 may comprise conventional melt processing polymers, such as, for example only, an elastomer such as PEBAX, with a melting temperature of about 130-175° C., which may be lower than the melting temperatures of the materials comprising core 36. Furthermore, outer layer 46 may comprise either a single section or multiple sections of tubing that are either butted together or overlapped with each other. The multiple segments, or layers, of outer layer material may be any length and/or hardness (durometer) allowing for flexibility of design, as known in the art. The durometer of outer layer 46 may be selected to match the mechanical properties of a portion of the medical device with which the second embodiment will be coupled. For example, it is known to use more flexible layer materials near the distal end portion of the medical device.

The assembly thus formed—core 36, coil 38, wiring 70, and outer layer 46—is then subjected to a reflow lamination process, which involves heating the assembly (e.g., in an oven designed for such processes) until the outer layer material flows and redistributes around the circumference, covering and enveloping coil 38, the exposed (not covered by coil 38) outer surface of core 36, and wiring 70. In one embodiment, the reflow process includes heating the device to about 450° F., though the reflow temperature may vary for other embodiments of the method. Core 36 preferably has a melting point higher than the reflow heating temperature, so it remains solid throughout the reflow step. The assembly is then cooled. After cooling, outer layer 46 may be a unitary jacket 46.

FIG. 9 is a cross-sectional end view of the second embodiment, taken substantially along line 9-9 in FIG. 8. Although FIG. 8 is also a cross-section, FIG. 9 shows the full circumference of the second embodiment. Heat-fused jacket 46 encompasses coil 38 and core 36. Comparing FIG. 9 (in which core 36 has undergone a fusion lamination process) to FIG. 4 (in which it has not), core 36 retains its pre-heating shape and size. Advantageously, core 36 may comprise a material, such as polyimide, that retains its shape at the temperature used for fusion of jacket 46, so the inner dimensions of sensor 24a are predictable so sensor 24a may reliably be inserted upon or threaded on medical devices such as MPS-enabled medical device 26.

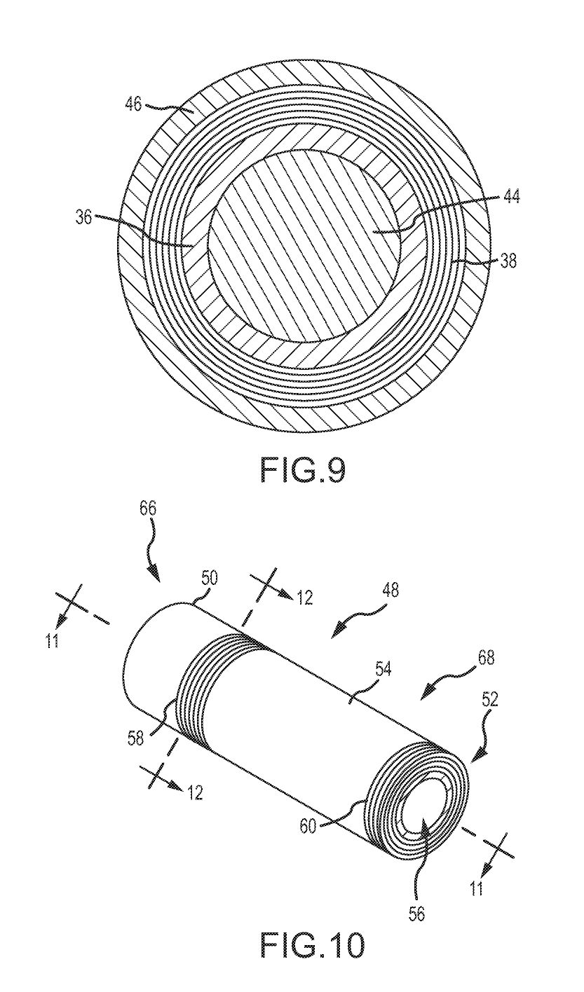

FIG. 10 is an isometric view of a medical device 48 having multiple electromagnetic coil positioning sensors incorporated therein. As described in the Background, one set of challenges in incorporating coil positioning sensors into certain medical devices involves maintaining an outer diameter dimension so as to retain cofunctionality with an outer sheath, as well as maintaining the dimensions and clearance of an innermost lumen to retain cofunctionality of the device with a guidewire. FIGS. 10-12 show an embodiment that meets these challenges, as described and illustrated below.

Medical device 48 may include an elongate body portion 50 with a proximal end portion 66, a distal end portion 68, an extreme distal end 52, and an outer surface 54. Elongate body portion 50 is configured to include a central lumen 56 extending from proximal end 66 to distal end 68. Lumen 56 is configured in size and shape to accommodate a guidewire, for example. Device 48 also includes a first electromagnetic field coil positioning sensor 58 and a second electromagnetic field coil positioning sensor 60. Coils 58 and 60 may be configured in design and function the same as sensors 24₁ and 24₂ or sensor 24a described above. Although second sensor 60 is shown at extreme distal end 52, the number and location of sensors on device 48 may vary according to device needs. In an alternate embodiment, body portion 50 may be solid, rather than hollow, and therefore may lack a central lumen.

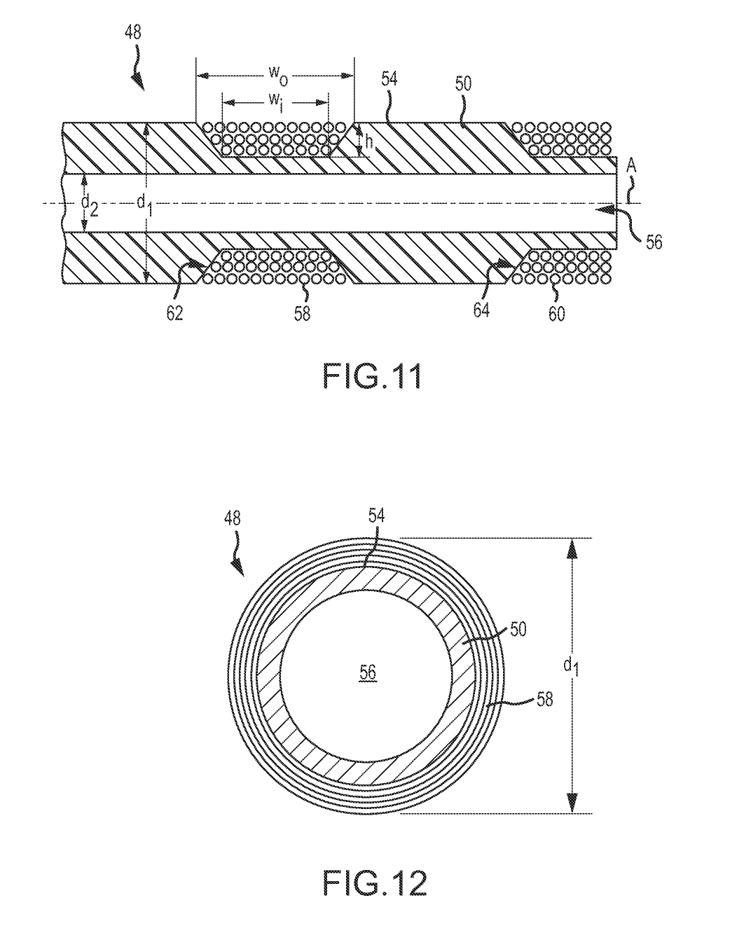

FIG. 11 is a cross-sectional side view of the third embodiment, taken substantially along line 11-11 in FIG. 10. Body 50 has an outer diameter d₁ and lumen 56 has an inner diameter d₂. Outer diameter d₁ may be taken with respect to outer surface 54. Outer surface 54 has a plurality of axially-spaced, circumferentially-extending grooves—shown in exemplary fashion with two such grooves 62 and 64—in which sensors 58, 60 may be respectively disposed. Grooves 62, 64 have a height h, and groove 62 has an inner width wᵢ (taken in the axial direction) and an outer width wₒ (also taken in the axial direction). Grooves 62, 64 may each have a winding surface for respective coils 58, 60. Groove 62 is bounded on axial ends thereof by angled sidewalls which form winding flanges. Groove 64 includes one such sidewall/winding flange. Coils 58, 60 may be wound directly in grooves 62, 64. In another embodiment, a pre-formed air coil (i.e., a coil without a core) or a coil formed on a core or other intermediate layer (such as the first embodiment of sensor 24a described above in conjunction with FIGS. 3-4) may be placed in a groove. Dimensions h, wᵢ, and wₒ may vary from groove to groove, and from device to device, so the trapezoidal shape of groove 62 and the step-like shape of groove 64 are exemplary only in nature. In addition, while coils 58, 60 are shown as substantially conforming to the shape of their respective grooves, this relationship need not be the case. Grooves 62, 64 may be created by various methods known in the art, such as, for example only, by mechanical micro-machining, laser machining, and micro-grinding. Although device 48 is shown with two grooves, any number of grooves, and any location of those grooves, may be employed depending on the needs for a particular device.

FIG. 12 is a cross-sectional view taken substantially along line 12-12 in FIG. 10. As shown, the maximum outside diameter (d₁) in the region of positioning sensor 58 does not exceed the maximum outside diameter (also d₁) in the main body portion. Likewise, central lumen 56 retains its nominal dimension and shape in the region of sensor 58, which is the same as in the main body portion.

The third embodiment advantageously may incorporate multiple sensors 58, 60 without increasing the outer diameter of the device, resulting in a substantially smooth outer surface with no increase in outer diameter. If such smoothness is desired, groove height h defines the desired thickness of a sensor in that groove, so that the outer diameter of device 48 is not increased by the sensor. In other words, where outer diameter d₁ defines an envelope radially-outwardly of a groove, the sensor disposed in that groove may remain within the envelope. In an exemplary embodiment, body portion 50 and sensors 58, 60 form a catheter. Because sensors 58, 60 may maintain the outer diameter of the catheter, device 48 advantageously may have a relatively smooth outer surface so that an outer sheath (not shown) or other medical device may be passed radially-outwardly of device 48. The sheath may have its own main lumen and inside diameter selected relative to the outside diameter of the catheter, allowing the catheter to pass through the sheath main lumen. Additionally, lumen 56 may retain its original dimension and shape, allowing continued co-functionality with a guidewire (not shown).

FIG. 13 is a schematic and block diagram of one exemplary embodiment of MPS 20, designated as an MPS 108, as also seen by reference to U.S. Pat. No. 7,386,339, referred to above, and portions of which are reproduced below, which generally describes, at least in part, the gMPS™ medical positioning system commercially offered by MediGuide Ltd. of Haifa, Israel and now owned by St. Jude Medical, Inc. It should be understood that variations are possible, for example, as also seen by reference to U.S. Pat. No. 6,233,476 entitled MEDICAL POSITIONING SYSTEM, also hereby incorporated by reference in its entirety. Another exemplary magnetic field-based MPS is the Carto™ system commercially available from Biosense Webster, and as generally shown and described in, for example, U.S. Pat. No. 6,498,944 entitled “Intrabody Measurement,” and U.S. Pat. No. 6,788,967 entitled “Medical Diagnosis, Treatment and Imaging Systems,” both of which are incorporated herein by reference in their entireties. Accordingly, the following description is exemplary only and not limiting in nature.

MPS system 110 includes a location and orientation processor 150, a transmitter interface 152, a plurality of look-up table units 154₁, 154₂ and 154₃, a plurality of digital to analog converters (DAC) 156₁, 156₂ and 156₃, an amplifier 158, a transmitter 160, a plurality of MPS sensors 162₁, 162₂, 162₃ and 162_{N}, a plurality of analog to digital converters (ADC) 164₁, 164₂, 164₃ and 164_{N} and a sensor interface 166.

Transmitter interface 152 is connected to location and orientation processor 150 and to look-up table units 154₁, 154₂ and 154₃. DAC units 156₁, 156₂ and 156₃ are connected to a respective one of look-up table units 154₁, 154₂ and 154₃ and to amplifier 158. Amplifier 158 is further connected to transmitter 160. Transmitter 160 is also marked TX. MPS sensors 162₁, 162₂, 162₃ and 162_{N} are further marked RX₁, RX₂, RX₃ and RX_{N}, respectively. Analog to digital converters (ADC) 164₁, 164₂, 164₃ and 164_{N} are respectively connected to sensors 162₁, 162₂, 162₃ and 162_{N} and to sensor interface 166. Sensor interface 166 is further connected to location and orientation processor 150.

Each of look-up table units 154₁, 154₂ and 154₃ produces a cyclic sequence of numbers and provides it to the respective DAC unit 156₁, 156₂ and 156₃, which in turn translates it to a respective analog signal. Each of the analog signals is respective of a different spatial axis. In the present example, look-up table 154₁ and DAC unit 156₁ produce a signal for the X axis, look-up table 154₂ and DAC unit 156₂ produce a signal for the Y axis and look-up table 154₃ and DAC unit 156₃ produce a signal for the Z axis.

DAC units 156₁, 156₂ and 156₃ provide their respective analog signals to amplifier 158, which amplifies and provides the amplified signals to transmitter 160. Transmitter 160 provides a multiple axis electromagnetic field, which can be detected by MPS sensors 162₁, 162₂, 162₃ and 162_{N}. Each of MPS sensors 162₁, 162₂, 162₃ and 162_{N} detects an electromagnetic field, produces a respective electrical analog signal and provides it to the respective ADC unit 164₁, 164₂, 164₃ and 164_{N} connected thereto. Each of the ADC units 164₁, 164₂, 164₃ and 164_{N} digitizes the analog signal fed thereto, converts it to a sequence of numbers and provides it to sensor interface 166, which in turn provides it to location and orientation processor 150. Location and orientation processor 150 analyzes the received sequences of numbers, thereby determining the location and orientation of each of the MPS sensors 162₁, 162₂, 162₃ and 162_{N}. Location and orientation processor 150 further determines distortion events and updates look-up tables 154₁, 154₂ and 154₃, accordingly.

It should be understood that system 10, particularly the main electronic control unit 12, as described above may include conventional processing apparatus known in the art, capable of executing pre-programmed instructions stored in an associated memory, all performing in accordance with the functionality described herein. Such an electronic control unit may further be of the type having both ROM, RAM, a combination of non-volatile and volatile (modifiable) memory so that any software may be stored and yet allow storage and processing of dynamically produced data and/or signals.

Although numerous embodiments of this invention have been described above with a certain degree of particularity, those skilled in the art could make numerous alterations to the disclosed embodiments without departing from the spirit or scope of this invention. All directional references (e.g., plus, minus, upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A positioning sensor assembly for use in a medical device comprising a heat-fused layer, the layer comprising a first material having a first melting temperature associated therewith, the positioning sensor assembly comprising:
a tubular core comprising a second material having a second melting temperature associated therewith that is higher than said first temperature, said core having a central through-bore extending along an axis between opposing axial ends of said core, said core further having a radially-outermost winding surface; and
an electrically conductive coil wound on said winding surface.

2. The assembly of claim 1 wherein said second material comprises a polymer.

3. The assembly of claim 2 wherein said second material comprises polyimide having a melting temperature of greater than 450° F.

4. The assembly of claim 3 wherein said heat-fused layer is radially-outwardly of said sensor in said medical device, said first material comprising an elastomer.

5. The assembly of claim 4 wherein said first material comprises PEBAX material having said first melting temperature of about 130-175° C.

6. The assembly of claim 3 wherein said core has a wall thickness of about 60 micrometers.

7. The assembly of claim 1, further comprising at least one wire coupled to said coil, said at least one wire extending axially-proximally from said coil.

8. The assembly of claim 7, wherein said coil is one of:
permanently fixed to said core; and
temporarily fixed to said core.

9. A medical device comprising:
a heat-fused layer comprising an elastomer having a first melting temperature associated therewith; and
a positioning sensor assembly comprising:
a tubular core comprising a polymer having a second melting temperature associated therewith that is higher than said first temperature, said core having a central through-bore extending along an axis between opposing axial ends of said core, said core further having a radially-outermost winding surface; and
an electrically conductive coil wound on said winding surface;
wherein said heat-fused layer is radially-outwardly of said sensor in said medical device.

10. A method of assembling a positioning sensor for a medical device, comprising:
providing a tubular core comprising a first material having a first melting temperature associated therewith, said core having a central through-bore extending along an axis between opposing axial ends of said core, said core further having a radially-outermost winding surface;
winding an electrically-conductive coil on said winding surface;
applying an outer layer to said coil and said core, said outer layer comprising a second material having a second melting temperature associated therewith that is lower than said first temperature; and
subjecting said core, said coil, and said outer layer to a reflow lamination process at a third temperature that is higher than said second temperature and lower than said first temperature.

11. The method of claim 10 wherein said first material comprises polyimide.

12. The method of claim 11 wherein said outer layer is radially-outwardly of said core and said coil in said medical device, said second material comprising an elastomer.

13. The method of claim 12 wherein said second material comprises PEBAX material having said second melting temperature of about 130-175° C.

14. The method of claim 10 wherein said third temperature is about 450° F.

15. A medical device configured for use with a medical positioning system (MPS), comprising:
an elongate body having an axis and an outer surface, said outer surface including a plurality of axially-spaced circumferentially-extending grooves, each of said grooves configured to receive a respective positioning sensor, said body having an outer diameter taken with respect to said outer surface; and
a plurality of sensors respectively disposed in a corresponding one of said grooves, said sensors each comprising a respective coil, each one of said sensors being configured relative to a corresponding one of said grooves so as to remain within an envelope defined by said outer diameter.

16. The device of claim 15 wherein said body further comprises a proximal end portion, a distal end portion, and a central lumen extending from said proximal end portion to said distal end portion.

17. The device of claim 16 wherein said central lumen is configured in at least size and shape to receive a guidewire.

18. The device of claim 15 wherein said body and said sensors form a catheter, said device further comprising a sheath disposed radially outwardly from said catheter, said sheath having a main lumen with an inside diameter associated therewith, said inside diameter being selected relative to said outside diameter of said catheter so as to permit said catheter to pass through said main lumen.

19. The device of claim 15 wherein at least one of said grooves has a winding surface, wherein said at least one of said grooves further has one or more sidewalls defining winding flanges, wherein said winding flanges are angled relative to said axis and relative to said winding surface.

20. The device of claim 15 wherein said distal end portion includes an extreme distal end, wherein at least one of said grooves is formed in said extreme distal end.
